(19) United States
(12) Patent Application Publication
(10) Pub. No.: US 2021/0259583 A1
(43) Pub. Date: Aug. 26, 2021

(54) MULTIPLEXING TECHNIQUES FOR INTERFERENCE REDUCTION IN TIME-CORRELATED SINGLE PHOTON COUNTING

(71) Applicant: HI LLC, Los Angeles, CA (US)

(72) Inventors: Sebastian Sorgenfrei, Playa Vista, CA (US); Ryan Field, Culver City, CA (US); Katherine Perdue, Los Angeles, CA (US); Jacob Dahle, Arlington, MA (US)

(21) Appl. No.: 17/176,307

(22) Filed: Feb. 16, 2021

Related U.S. Application Data
(60) Provisional application No. 63/040,773, filed on Jun. 18, 2020.
(60) Provisional application No. 62/992,491, filed on Mar. 20, 2020.
(60) Provisional application No. 62/979,866, filed on Feb. 21, 2020.

(51) Int. Cl. A61B 5/1455; A61B 5/00

(52) U.S. Cl. CPC A61B 5/1455; A61B 2562/046; A61B 5/6803; A61B 5/4064

(57) ABSTRACT

An optical measurement system includes a first wearable module comprising a first source configured to emit a first light pulse sequence comprising a plurality of light pulses and a first plurality of detectors configured to detect photons from the first light pulse sequence. The system further includes a second wearable module comprising a second source configured to emit a second light pulse sequence comprising a plurality of light pulses and that is time interleaved with the first light pulse sequence, and a second plurality of detectors configured to detect photons from the second light pulse sequence. The system further includes a control circuit configured to control light pulses emitted by the sources in accordance with time and/or frequency division multiplexing heuristics.

### RELATED APPLICATIONS

The present application claims priority under 35 U.S.C. § 119(e) to U.S. Provisional Patent Application No. 63/040,773, filed on Jun. 18, 2020, and to U.S. Provisional Patent Application No. 62/992,491, filed on Mar. 20, 2020, and to U.S. Provisional Patent Application No. 62/979,866, filed on Feb. 21, 2020. These applications are incorporated herein by reference in their respective entireties.

### BACKGROUND INFORMATION

Detecting neural activity in the brain (or any other turbid medium) is useful for medical diagnostics, imaging, neuroengineering, brain-computer interfacing, and a variety of other diagnostic and consumer-related applications. For example, it may be desirable to detect neural activity in the brain of a user to determine if a particular region of the brain has been impacted by reduced blood irrigation, a hemorrhage, or any other type of damage. As another example, it may be desirable to detect neural activity in the brain of a user and computationally decode the detected neural activity into commands that can be used to control various types of consumer electronics (e.g., by controlling a cursor on a computer screen, changing channels on a television, turning lights on, etc.).

Neural activity and other attributes of the brain may be determined or inferred by measuring responses of tissue within the brain to light pulses. One technique to measure such responses is time-correlated single-photon counting (TCSPC). Time-correlated single-photon counting detects single photons and measures a time of arrival of the photons with respect to a reference signal (e.g., a light source). By repeating the light pulses, TCSPC may accumulate a sufficient number of photon events to statistically determine a histogram representing the distribution of detected photons. Based on the histogram of photon distribution, the response of tissue to light pulses may be determined in order to study the detected neural activity and/or other attributes of the brain.

A photodetector capable of detecting a single photon (i.e., a single particle of optical energy) is an example of a non-invasive detector that can be used in an optical measurement system to detect neural activity within the brain. An exemplary photodetector is implemented by a semiconductor-based single-photon avalanche diode (SPAD), which is capable of capturing individual photons with very high time-of-arrival resolution (a few tens of picoseconds).

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary optical measurement system.

FIG. 2 illustrates an exemplary detector architecture.

FIG. 3 illustrates an exemplary timing diagram for performing an optical measurement operation using an optical measurement system.

FIG. 4 illustrates a graph of an exemplary temporal point spread function that may be generated by an optical measurement system in response to a light pulse.

FIG. 5 shows an exemplary non-invasive wearable brain interface system.

FIG. 6 shows an exemplary wearable module assembly.

FIG. 7 shows an implementation of the wearable module assembly of FIG. 6.

FIGS. 8-9 show exemplary time division multiplexing heuristics.

FIGS. 10-11 show exemplary frequency division multiplexing heuristics.

FIG. 12 illustrates an exemplary phase locked loop (PLL) circuit based architecture.

FIG. 13 shows an exemplary composition configuration.

FIGS. 14-19 illustrate embodiments of a wearable device that includes elements of the optical detection systems described herein.

FIG. 20 illustrates an exemplary computing device.

FIGS. 21-22 illustrate exemplary methods.

### DETAILED DESCRIPTION

Multiplexing techniques for interference reduction in time-correlated single photon counting are described herein.

Diffuse optical imaging technologies, such as optical coherence tomography (OCT), high-density diffuse optical tomography (HD-DOT) and functional near infrared spectroscopy (fNIRS), have been used to investigate a hemodynamic response using near-infrared or visible light radiation. Unfortunately, conventional optical measurement systems that implement these optical measurement techniques have poor temporal and/or spatial resolution and do not provide sufficient head coverage for a user.

In contrast, the systems, circuits, and methods described herein may be configured to detect optical parameters, such as absorption in a diffuse medium, with relatively good temporal (e.g., less than 100 milliseconds (ms)) and spatial (e.g., less than 10 millimeters (mm)) resolution while maximizing signal to noise ratio. This is achieved by generating short (e.g., 10-2000 picoseconds (ps)) and time interleaved light pulses at one or more wavelengths that are synchronized and then recording a response at multiple time-of-flight detectors with varying separation with precise timing. To maximize signal-to-noise ratio, the light pulses generated by neighboring light sources (or simply “sources”) may be time division multiplexed (offset in time) or frequency division multiplexed (offset in frequency). This may ensure that interference and/or crosstalk between neighboring sources and detectors is minimized, which may lead to an optimized signal-to-noise ratio.

An exemplary application of the present systems, circuits, and methods described herein is to non-invasively measure the concentrations of oxygenated (O2-Hb) and deoxygenated (HHb) hemoglobin in brain tissue by dynamically measuring absorption properties of the brain. To do this, an optical measurement system may use multiple sources (e.g., laser diodes or LEDs) and detectors (e.g., avalanche photon detectors with a fast analog-to-digital converter or a single photon avalanche diode with a time-to-digital converter) to obtain an accurate measurement of the oxygenated and deoxygenated hemoglobin concentrations at various locations on the brain. In some examples, multiple wavelengths may be used because the tissue absorption for oxygenated and deoxygenated hemoglobin is dependent on wavelength.

These and other advantages and benefits of the present systems, circuits, and methods are described more fully herein.

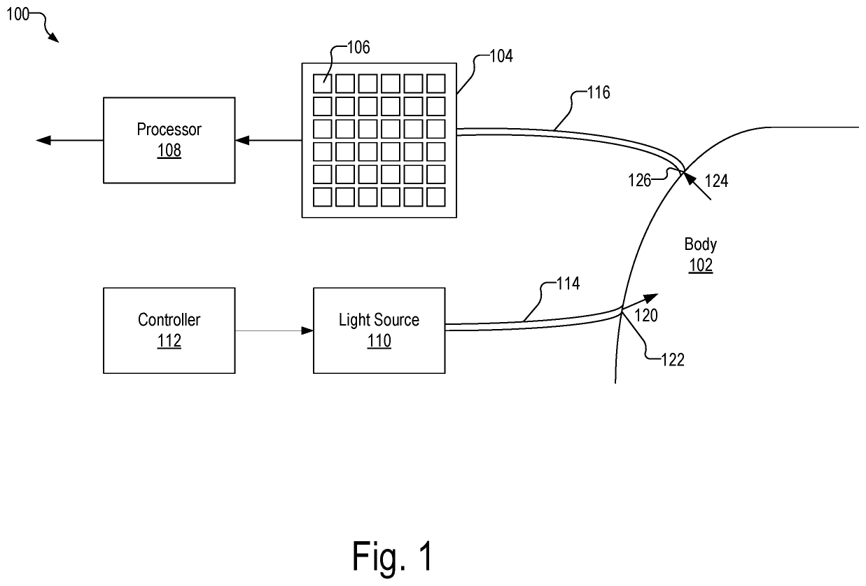

FIG. 1 shows an exemplary optical measurement system 100 configured to perform an optical measurement operation with respect to a body 102. Optical measurement system 100 may, in some examples, be portable and/or wearable by a user.

In some examples, optical measurement operations performed by optical measurement system 100 are associated with a time domain-based optical measurement technique. Example time domain-based optical measurement techniques include, but are not limited to, TCSPC, time domain near infrared spectroscopy (TD-NIRS), time domain diffusive correlation spectroscopy (TD-DCS), and time domain Digital Optical Tomography (TD-DOT).

As shown, optical measurement system 100 includes a detector 104 that includes a plurality of individual photodetectors (e.g., photodetector 106), a processor 108 coupled to detector 104, a light source 110, a controller 112, and optical conduits 114 and 116 (e.g., light pipes). However, one or more of these components may not, in certain embodiments, be considered to be a part of optical measurement system 100. For example, in implementations where optical measurement system 100 is wearable by a user, processor 108 and/or controller 112 may in some embodiments be separate from optical measurement system 100 and not configured to be worn by the user.

Detector 104 may include any number of photodetectors 106 as may serve a particular implementation, such as 2ⁿ photodetectors (e.g., 256, 512, . . . , 16384, etc.), where n is an integer greater than or equal to one (e.g., 4, 5, 8, 10, 11, 14, etc.). Photodetectors 106 may be arranged in any suitable manner.

Photodetectors 106 may each be implemented by any suitable circuit configured to detect individual photons of light incident upon photodetectors 106. For example, each photodetector 106 may be implemented by a single photon avalanche diode (SPAD) circuit and/or other circuitry as may serve a particular implementation.

Processor 108 may be implemented by one or more physical processing (e.g., computing) devices. In some examples, processor 108 may execute instructions (e.g., software) configured to perform one or more of the operations described herein.

Light source 110 may be implemented by any suitable component configured to generate and emit light. For example, light source 110 may be implemented by one or more laser diodes, distributed feedback (DFB) lasers, super luminescent diodes (SLDs), light emitting diodes (LEDs), diode-pumped solid-state (DPSS) lasers, super luminescent light emitting diodes (sLEDs), vertical-cavity surface-emitting lasers (VCSELs), titanium sapphire lasers, micro light emitting diodes (mLEDs), and/or any other suitable laser or light source. In some examples, the light emitted by light source 110 is high coherence light (e.g., light that has a coherence length of at least 5 centimeters) at a predetermined center wavelength.

Light source 110 is controlled by controller 112, which may be implemented by any suitable computing device (e.g., processor 108), integrated circuit, and/or combination of hardware and/or software as may serve a particular implementation. In some examples, controller 112 is configured to control light source 110 by turning light source 110 on and off and/or setting an intensity of light generated by light source 110. Controller 112 may be manually operated by a user, or may be programmed to control light source 110 automatically.

Light emitted by light source 110 travels via an optical conduit 114 (e.g., a light pipe, a single-mode optical fiber, and/or or a multi-mode optical fiber) to body 102 of a subject. Body 102 may include any suitable turbid medium. For example, in some implementations, body 102 is a head or any other body part of a human or other animal. Alternatively, body 102 may be a non-living object. For illustrative purposes, it will be assumed in the examples provided herein that body 102 is a human head.

As indicated by arrow 120, the light emitted by light source 110 enters body 102 at a first location 122 on body 102. Accordingly, a distal end of optical conduit 114 may be positioned at (e.g., right above, in physical contact with, or physically attached to) first location 122 (e.g., to a scalp of the subject). In some examples, the light may emerge from optical conduit 114 and spread out to a certain spot size on body 102 to fall under a predetermined safety limit. At least a portion of the light indicated by arrow 120 may be scattered within body 102.

As used herein, “distal” means nearer, along the optical path of the light emitted by light source 110 or the light received by detector 104, to the target (e.g., within body 102) than to light source 110 or detector 104. Thus, the distal end of optical conduit 114 is nearer to body 102 than to light source 110, and the distal end of optical conduit 116 is nearer to body 102 than to detector 104. Additionally, as used herein, “proximal” means nearer, along the optical path of the light emitted by light source 110 or the light received by detector 104, to light source 110 or detector 104 than to body 102. Thus, the proximal end of optical conduit 114 is nearer to light source 110 than to body 102, and the proximal end of optical conduit 116 is nearer to detector 104 than to body 102.

As shown, the distal end of optical conduit 116 (e.g., a light pipe, a light guide, a waveguide, a single-mode optical fiber, and/or a multi-mode optical fiber) is positioned at (e.g., right above, in physical contact with, or physically attached to) output location 126 on body 102. In this manner, optical conduit 116 may collect at least a portion of the scattered light (indicated as light 124) as it exits body 102 at location 126 and carry light 124 to detector 104. Light 124 may pass through one or more lenses and/or other optical elements (not shown) that direct light 124 onto each of the photodetectors 106 included in detector 104.

Photodetectors 106 may be connected in parallel in detector 104. An output of each of photodetectors 106 may be accumulated to generate an accumulated output of detector 104. Processor 108 may receive the accumulated output and determine, based on the accumulated output, a temporal distribution of photons detected by photodetectors 106. Processor 108 may then generate, based on the temporal distribution, a histogram representing a light pulse response of a target (e.g., brain tissue, blood flow, etc.) in body 102. Example embodiments of accumulated outputs are described herein.

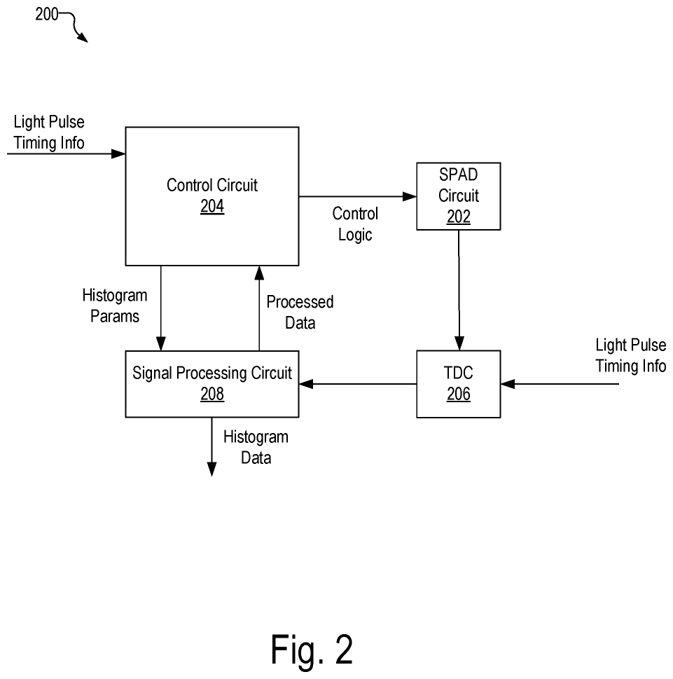

FIG. 2 illustrates an exemplary detector architecture 200 that may be used in accordance with the systems and methods described herein. As shown, architecture 200 includes a SPAD circuit 202 that implements photodetector 106, a control circuit 204, a time-to-digital converter (TDC) 206, and a signal processing circuit 208. Architecture 200 may include additional or alternative components as may serve a particular implementation.

In some examples, SPAD circuit 202 includes a SPAD and a fast gating circuit configured to operate together to detect a photon incident upon the SPAD. As described herein, SPAD circuit 202 may generate an output when SPAD circuit 202 detects a photon.

The fast gating circuit included in SPAD circuit 202 may be implemented in any suitable manner. For example, the fast gating circuit may include a capacitor that is pre-charged with a bias voltage before a command is provided to arm the SPAD. Gating the SPAD with a capacitor instead of with an active voltage source, such as is done in some conventional SPAD architectures, has a number of advantages and benefits. For example, a SPAD that is gated with a capacitor may be armed practically instantaneously compared to a SPAD that is gated with an active voltage source. This is because the capacitor is already charged with the bias voltage when a command is provided to arm the SPAD. This is described more fully in U.S. Pat. Nos. 10,158,038 and 10,424,683, which are incorporated herein by reference in their respective entireties.

In some alternative configurations, SPAD circuit 202 does not include a fast gating circuit. In these configurations, the SPAD included in SPAD circuit 202 may be gated in any suitable manner.

Control circuit 204 may be implemented by an application specific integrated circuit (ASIC) or any other suitable circuit configured to control an operation of various components within SPAD circuit 202. For example, control circuit 204 may output control logic that puts the SPAD included in SPAD circuit 202 in either an armed or a disarmed state.

In some examples, control circuit 204 may control a gate delay, which specifies a predetermined amount of time control circuit 204 is to wait after an occurrence of a light pulse (e.g., a laser pulse) to put the SPAD in the armed state. To this end, control circuit 204 may receive light pulse timing information, which indicates a time at which a light pulse occurs (e.g., a time at which the light pulse is applied to body 102). Control circuit 204 may also control a programmable gate width, which specifies how long the SPAD is kept in the armed state before being disarmed.

Control circuit 204 is further configured to control signal processing circuit 208. For example, control circuit 204 may provide histogram parameters (e.g., time bins, number of light pulses, type of histogram, etc.) to signal processing circuit 208. Signal processing circuit 208 may generate histogram data in accordance with the histogram parameters. In some examples, control circuit 204 is at least partially implemented by controller 112.

TDC 206 is configured to measure a time difference between an occurrence of an output pulse generated by SPAD circuit 202 and an occurrence of a light pulse. To this end, TDC 206 may also receive the same light pulse timing information that control circuit 204 receives. TDC 206 may be implemented by any suitable circuitry as may serve a particular implementation.

Signal processing circuit 208 is configured to perform one or more signal processing operations on data output by TDC 206. For example, signal processing circuit 208 may generate histogram data based on the data output by TDC 206 and in accordance with histogram parameters provided by control circuit 204. To illustrate, signal processing circuit 208 may generate, store, transmit, compress, analyze, decode, and/or otherwise process histograms based on the data output by TDC 206. In some examples, signal processing circuit 208 may provide processed data to control circuit 204, which may use the processed data in any suitable manner. In some examples, signal processing circuit 208 is at least partially implemented by processor 108.

In some examples, each photodetector 106 (e.g., SPAD circuit 202) may have a dedicated TDC 206 associated therewith. For example, for an array of N photodetectors 106, there may be a corresponding array of N TDCs 206. Alternatively, a single TDC 206 may be associated with multiple photodetectors 106. Likewise, a single control circuit 204 and a single signal processing circuit 208 may be provided for a one or more photodetectors 106 and/or TDCs 206.

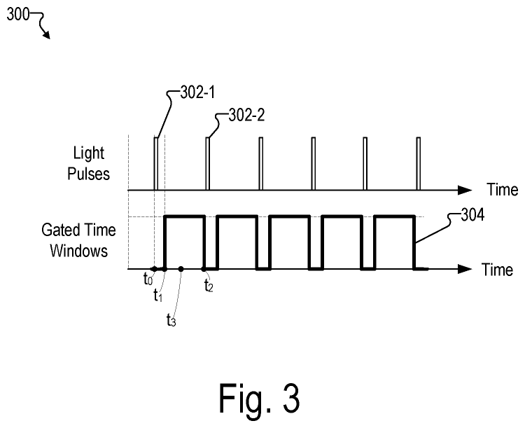

FIG. 3 illustrates an exemplary timing diagram 300 for performing an optical measurement operation using optical measurement system 100. Optical measurement system 100 may be configured to perform the optical measurement operation by directing light pulses (e.g., laser pulses) toward a target within a body (e.g., body 102). The light pulses may be short (e.g., 10-2000 picoseconds (ps)) and repeated at a high frequency (e.g., between 100,000 hertz (Hz) and 100 megahertz (MHz)). The light pulses may be scattered by the target and then detected by optical measurement system 100. Optical measurement system 100 may measure a time relative to the light pulse for each detected photon. By counting the number of photons detected at each time relative to each light pulse repeated over a plurality of light pulses, optical measurement system 100 may generate a histogram that represents a light pulse response of the target (e.g., a temporal point spread function (TPSF)). The terms histogram and TPSF are used interchangeably herein to refer to a light pulse response of a target.

For example, timing diagram 300 shows a sequence of light pulses 302 (e.g., light pulses 302-1 and 302-2) that may be applied to the target (e.g., tissue within a brain of a user, blood flow, a fluorescent material used as a probe in a body of a user, etc.). Timing diagram 300 also shows a pulse wave 304 representing predetermined gated time windows (also referred as gated time periods) during which photodetectors 106 are gated ON to detect photons. Referring to light pulse 302-1, light pulse 302-1 is applied at a time to. At a time t₁, a first instance of the predetermined gated time window begins. Photodetectors 106 may be armed at time t₁, enabling photodetectors 106 to detect photons scattered by the target during the predetermined gated time window. In this example, time t₁ is set to be at a certain time after time to, which may minimize photons detected directly from the laser pulse, before the laser pulse reaches the target. However, in some alternative examples, time t₁ is set to be equal to time t₀.

At a time t₂, the predetermined gated time window ends. In some examples, photodetectors 106 may be disarmed at time t₂. In other examples, photodetectors 106 may be reset (e.g., disarmed and re-armed) at time t₂ or at a time subsequent to time t₂. During the predetermined gated time window, photodetectors 106 may detect photons scattered by the target. Photodetectors 106 may be configured to remain armed during the predetermined gated time window such that photodetectors 106 maintain an output upon detecting a photon during the predetermined gated time window. For example, a photodetector 106 may detect a photon at a time t₃, which is during the predetermined gated time window between times t₁ and t₂. The photodetector 106 may be configured to provide an output indicating that the photodetector 106 has detected a photon. The photodetector 106 may be configured to continue providing the output until time t₂, when the photodetector may be disarmed and/or reset. Optical measurement system 100 may generate an accumulated output from the plurality of photodetectors. Optical measurement system 100 may sample the accumulated output to determine times at which photons are detected by photodetectors 106 to generate a TPSF.

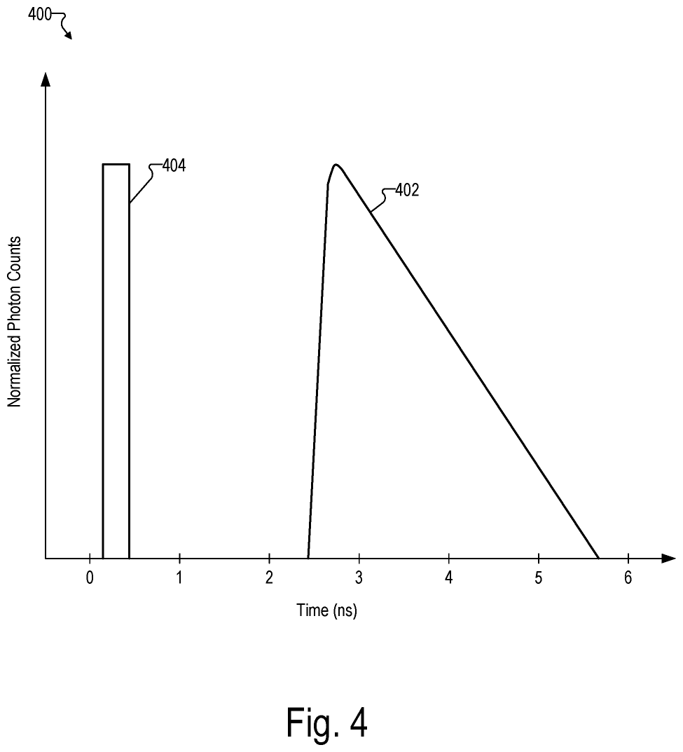

FIG. 4 illustrates a graph 400 of an exemplary TPSF 402 that may be generated by optical measurement system 100 in response to a light pulse 404 (which, in practice, represents a plurality of light pulses). Graph 400 shows a normalized count of photons on a y-axis and time bins on an x-axis. As shown, TPSF 402 is delayed with respect to a temporal occurrence of light pulse 404. In some examples, the number of photons detected in each time bin subsequent to each occurrence of light pulse 404 may be aggregated (e.g., integrated) to generate TPSF 402. TPSF 402 may be analyzed and/or processed in any suitable manner to determine or infer detected neural activity.

Optical measurement system 100 may be implemented by or included in any suitable device. For example, optical measurement system 100 may be included, in whole or in part, in a non-invasive wearable device (e.g., a headpiece) that a user may wear to perform one or more diagnostic, imaging, analytical, and/or consumer-related operations. The non-invasive wearable device may be placed on a user's head or other part of the user to detect neural activity. In some examples, such neural activity may be used to make behavioral and mental state analysis, awareness and predictions for the user.

Mental state described herein refers to the measured neural activity related to physiological brain states and/or mental brain states, e.g., joy, excitement, relaxation, surprise, fear, stress, anxiety, sadness, anger, disgust, contempt, contentment, calmness, focus, attention, approval, creativity, positive or negative reflections/attitude on experiences or the use of objects, etc. Further details on the methods and systems related to a predicted brain state, behavior, preferences, or attitude of the user, and the creation, training, and use of neuromes can be found in U.S. Provisional Patent Application No. 63/047,991, filed Jul. 3, 2020. Exemplary measurement systems and methods using biofeedback for awareness and modulation of mental state are described in more detail in U.S. patent application Ser. No. 16/364,338, filed Mar. 26, 2019, published as US2020/0196932A1. Exemplary measurement systems and methods used for detecting and modulating the mental state of a user using entertainment selections, e.g., music, film/video, are described in more detail in U.S. patent application Ser. No. 16/835,972, filed Mar. 31, 2020, published as US2020/0315510A1. Exemplary measurement systems and methods used for detecting and modulating the mental state of a user using product formulation from, e.g., beverages, food, selective food/drink ingredients, fragrances, and assessment based on product-elicited brain state measurements are described in more detail in U.S. patent application Ser. No. 16/853,614, filed Apr. 20, 2020, published as US2020/0337624A1. Exemplary measurement systems and methods used for detecting and modulating the mental state of a user through awareness of priming effects are described in more detail in U.S. patent application Ser. No. 16/885,596, filed May 28, 2020, published as US2020/0390358A1. These applications and corresponding U.S. publications are incorporated herein by reference in their entirety.

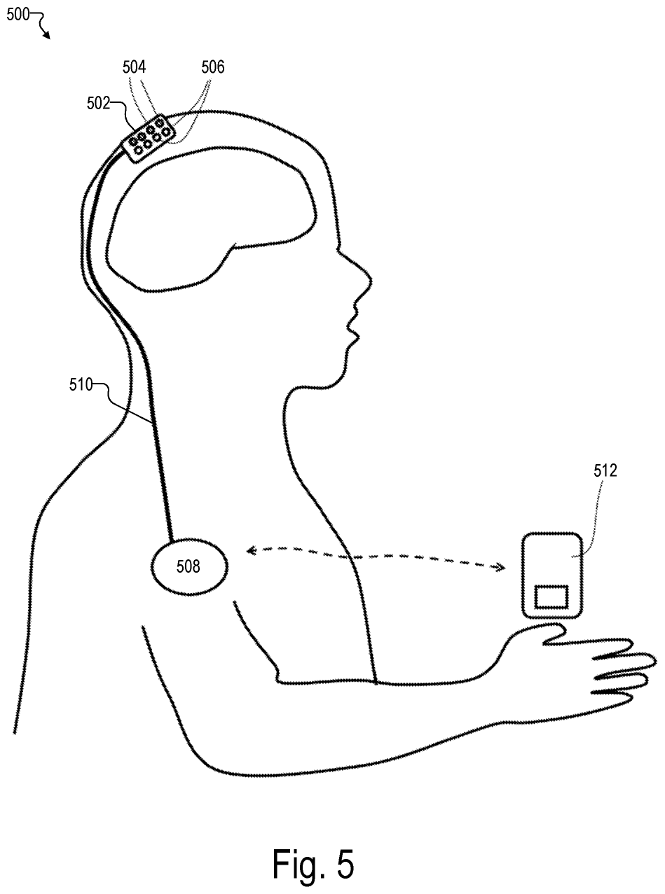

To illustrate, FIG. 5 shows an exemplary non-invasive wearable brain interface system 500 (“brain interface system 500”) that implements optical measurement system 100 (shown in FIG. 1). As shown, brain interface system 500 includes a head-mountable component 502 configured to be attached to a user's head. Head-mountable component 502 may be implemented by a cap shape that is worn on a head of a user. Alternative implementations of head-mountable component 502 include helmets, beanies, headbands, other hat shapes, or other forms conformable to be worn on a user's head, etc. Head-mountable component 502 may be made out of any suitable cloth, soft polymer, plastic, hard shell, and/or any other suitable material as may serve a particular implementation. Examples of headgears used with wearable brain interface systems are described more fully in U.S. Pat. No. 10,340,408, incorporated herein by reference in its entirety.

Head-mountable component 502 includes a plurality of detectors 504, which may implement or be similar to detector 104, and a plurality of light sources 506, which may be implemented by or be similar to light source 110. It will be recognized that in some alternative embodiments, head-mountable component 502 may include a single detector 504 and/or a single light source 506.

Brain interface system 500 may be used for controlling an optical path to the brain and for transforming photodetector measurements into an intensity value that represents an optical property of a target within the brain. Brain interface system 500 allows optical detection of deep anatomical locations beyond skin and bone (e.g., skull) by extracting data from photons originating from light source 506 and emitted to a target location within the user's brain, in contrast to conventional imaging systems and methods (e.g., optical coherence tomography (OCT)), which only image superficial tissue structures or through optically transparent structures.

Brain interface system 500 may further include a processor 508 configured to communicate with (e.g., control and/or receive signals from) detectors 504 and light sources 506 by way of a communication link 510. Communication link 510 may include any suitable wired and/or wireless communication link. Processor 508 may include any suitable housing and may be located on the user's scalp, neck, shoulders, chest, or arm, as may be desirable. In some variations, processor 508 may be integrated in the same assembly housing as detectors 504 and light sources 506.

As shown, brain interface system 500 may optionally include a remote processor 512 in communication with processor 508. For example, remote processor 512 may store measured data from detectors 504 and/or processor 508 from previous detection sessions and/or from multiple brain interface systems (not shown). Power for detectors 504, light sources 506, and/or processor 508 may be provided via a wearable battery (not shown). In some examples, processor 508 and the battery may be enclosed in a single housing, and wires carrying power signals from processor 508 and the battery may extend to detectors 504 and light sources 506. Alternatively, power may be provided wirelessly (e.g., by induction).

In some alternative embodiments, head mountable component 502 does not include individual light sources. Instead, a light source configured to generate the light that is detected by a detector 504 may be included elsewhere in brain interface system 500. For example, a light source may be included in processor 508 and coupled to head mountable component 502 through optical connections.

Optical measurement system 100 may alternatively be included in a non-wearable device (e.g., a medical device and/or consumer device that is placed near the head or other body part of a user to perform one or more diagnostic, imaging, and/or consumer-related operations). Optical measurement system 100 may alternatively be included in a sub-assembly enclosure of a wearable invasive device (e.g., an implantable medical device for brain recording and imaging).

In some examples, a wearable device implementing any of the features described herein may be modular in that one or more components of the optical measurement system may be removed, changed out, or otherwise modified as may serve a particular implementation. Additionally or alternatively, a wearable device implementing any of the features described may be modular such that one or more components of the wearable device may be housed in a separate housing (e.g., module) and/or may be movable relative to other components. Exemplary modular wearable devices are described in more detail in U.S. Provisional Patent Application No. 63/081,754, filed Sep. 22, 2020, U.S. Provisional Patent Application No. 63/038,459, filed Jun. 12, 2020, U.S. Provisional Patent Application No. 63/038,468, filed Jun. 12, 2020, U.S. Provisional Patent Application No. 63/038,481, filed Jun. 12, 2020, and U.S. Provisional Patent Application No. 63/064,688, filed Aug. 12, 2020, which applications are incorporated herein by reference in their respective entireties.

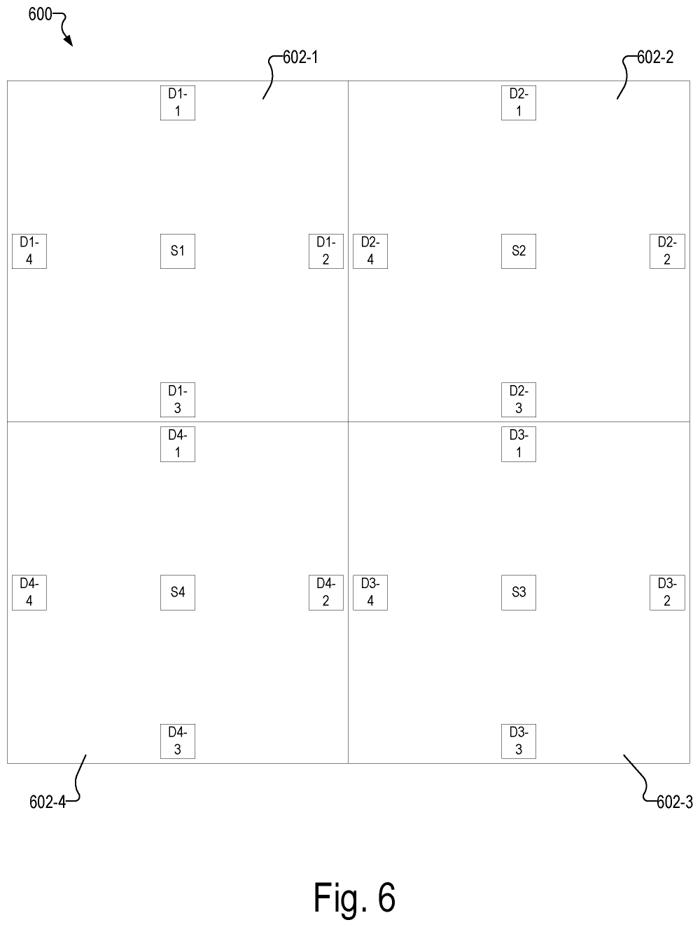

To illustrate, FIG. 6 shows an exemplary wearable module assembly 600 (“assembly 600”) that implements one or more of the optical measurement features described herein. Assembly 600 may be worn on the head or any other suitable body part of the user. As shown, assembly 600 may include a plurality of modules 602 (e.g., modules 602-1 through 602-4). While four modules 602 are shown to be included in assembly 600 in FIG. 6, in alternative configurations, any number of modules 602 (e.g., a single module up to sixteen or more modules) may be included in assembly 600. Moreover, while modules 602 are shown to be adjacent to and touching one another, modules 602 may alternatively be spaced apart from one another (e.g., in implementations where modules 602 are configured to be inserted into individual slots or cutouts of headgear). Moreover, while modules 602 are shown to be rectangular, modules 602 may alternatively have any other suitable geometry (e.g., in the shape of a pentagon, hexagon, octagon, square, circle, triangle, free-form shape, etc.).

Each module 602 includes a source and a plurality of time-of-flight detectors. For example, module 602-1 includes a source S1 surrounded by four detectors D1-1, D1-2, D1-3, and D1-4. Likewise, module 602-2 includes a source S2 surrounded by four detectors D2-1, D2-2, D2-3, and D2-4, module 602-3 includes a source S3 surrounded by four detectors D3-1, D3-2, D3-3, and D3-4, and module 602-4 includes a source S4 surrounded by four detectors D4-1, D4-2, D4-3, and D4-4.

Each source depicted in FIG. 6 may be implemented by one or more light sources similar to light source 110. For example, each source may be implemented by a laser diode, an LED, and/or any others suitable type of light source.

Each detector depicted in FIG. 6 may implement or be similar to detector 104 and may include a plurality of photodetectors (e.g., SPADs or avalanche photon detectors) as well as other circuitry (e.g., TDCs). As shown, detectors on each module 602 are arranged around and substantially equidistant from the module's source. In other words, the spacing between a light source (e.g., a distal end portion of a light source optical conduit) and the detectors (e.g., distal end portions of optical conduits for each detector) may be maintained at the same fixed distance on each module to ensure homogeneous coverage over specific areas and to facilitate processing of the detected signals. The fixed spacing also provides consistent spatial (lateral and depth) resolution across the target area of interest, e.g., brain tissue. Moreover, maintaining a known distance between the source and the detectors allows subsequent processing of the detected signals to infer spatial (e.g., depth localization, inverse modeling) information about the detected signals. Detectors 606 may be alternatively disposed as may serve a particular implementation.

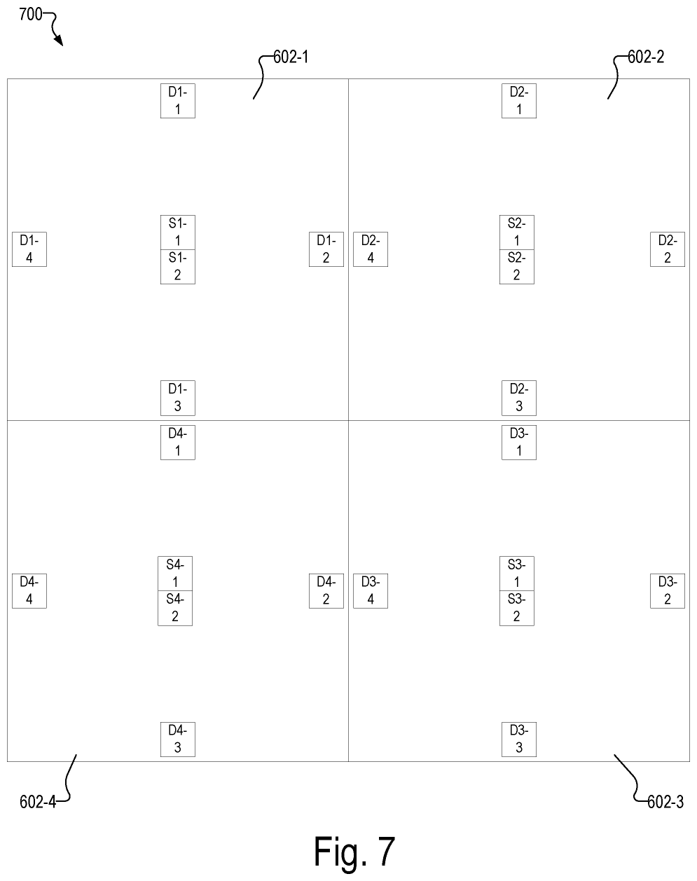

FIG. 7 shows an alternative implementation 700 of assembly 600 in which each module 602 includes multiple sources. For example, as shown, module 602-1 includes sources S1-1 and S1-2, module 602-2 includes sources S2-1 and S2-2, module 602-3 includes sources S3-1 and S3-2, and module 602-4 includes sources S4-1 and S4-2. As shown, each pair of sources may be co-located (e.g., right next to each other) on their respective module 602. In this configuration, each source in each pair of sources may operate at a different wavelength so that the concentrations of oxygenated and deoxygenated hemoglobin (which are at different wavelengths) can be measured. For example, sources S1-1, S2-1, S3-1, and S4-1 may each operate at a first wavelength (e.g., by emitting a first light pulse sequence having light pulses each having the first wavelength) and sources S1-2, S2-2, S3-2, and S4-2 may each operate at a second wavelength that is different than the first wavelength (e.g., by emitting a second light pulse sequence having light pulses each having the second wavelength).

While the examples described herein assume that each module 602 includes one or two sources and four detectors, it will be recognized that assembly 600 may include any number of sources and any number of detectors as may serve a particular implementation.

Each detector on a module 602 is configured to detect photons from a light pulse sequence emitted by a source located on the same module 602 as the light pulses included in the sequence are scattered by a target (e.g., a brain) within a body (e.g., a user's head). By so doing, each detector may detect a response (e.g., a brain hemodynamic response, a brain hemoglobulin response, and/or any other suitable type of response) that occurs when a particular light pulse is emitted by the source. Hence, each detector may detect a plurality of responses corresponding to a light pulse sequence (e.g., one response per light pulse included in the light pulse sequence).

For example, referring to FIG. 6, detectors D1-1, D1-2, D1-3, and D1-4 are each configured to detect photons from a light pulse sequence emitted by source S1. As another example, referring to FIG. 7, detectors D1-1, D1-2, D1-3, and D1-4 are each configured to detect photons from a light pulse sequence emitted by source S1-1 and a light pulse sequence emitted by source S1-2.

In some cases, a detector on a module 602 may also detect photons from a light pulse sequence emitted by a source located on a neighboring module 602. This may be due to a proximity of the detector to the source located on the neighboring module 602. For example, detector D1-2 of module 602-1 is relatively close to source S2 of module 602-2. Due to this proximity, detector D1-2 may detect photons from a light pulse sequence emitted by source S2, in addition to detecting photons from the light pulse sequence emitted by source S1. The remaining detectors D1-1, D1-3, and D1-4 on module 602-1 may, in some configurations, be too far from source S2 to detect photons from the light pulse sequence emitted by source S2. However, these remaining detectors may detect photons from light pulses sequences emitted by sources on other nearest neighbor modules 602 to module 602-1. For example, detector D1-3 may be close enough to source S4 of module 602-4 to detect photons from a light pulse sequence emitted by source S4.

A light pulse emitted from a source (e.g., S2) on a module (e.g., module 602-2) may, in some cases, interfere with a light pulse emitted from a source (e.g., S1) on a neighboring module (e.g., module 602-1). For example, if the two light pulses temporally overlap, a detector (e.g., detector D1-2) that is near enough to both sources may concurrently detect responses for both light pulses. This interference may skew or otherwise degrade the response detected by the detector.

Hence, the systems, circuits, and methods described herein may utilize time division multiplexing and/or frequency division multiplexing to minimize or eliminate such interference between light pulses emitted by sources on neighboring modules 602 and thereby maximize signal-to-noise ratio of the responses detected by detectors on the neighboring modules 602.

For example, a control circuit (e.g., control circuit 204 or any other suitable controller) may be configured to implement time division multiplexing by controlling a timing of the light pulse sequences emitted by each of the sources within assembly 600.

To illustrate, an optical measurement system may include first and second wearable modules (e.g., modules 602-1 and 602-2). The first wearable module may include a first source (e.g., source S1) configured to emit a first light pulse sequence comprising a plurality of light pulses and a first plurality of detectors (e.g., detectors D1-1, D1-2, D1-3, and D1-4) configured to detect photons from the first light pulse sequence. The second wearable module may include a second source (e.g., source S2) configured to emit a second light pulse sequence comprising a plurality of light pulses and that is time interleaved with the first light pulse sequence and a second plurality of detectors (e.g., detectors D2-1, D2-2, D2-3, and D2-4) configured to detect photons from the second light pulse sequence. A control circuit is configured to implement time division multiplexing by preventing the light pulses of the second light pulse sequence from temporally overlapping with the light pulses of the first light pulse sequence. This may be performed by directing the first source (e.g., source S1) to begin emitting the first light pulse sequence at a first time and directing the second source (e.g., source S2) to begin emitting the second light pulse sequence at a second time that is temporally offset from the first time.

Continuing with this example, the first plurality of detectors (e.g., within module 602-1) may include a first detector (e.g., detector D1-2) spatially closer to the second source (e.g., source S2) than any other detector included in the first plurality of detectors. The second plurality of detectors (e.g., within module 602-2) may likewise include a second detector (e.g., detector D2-4) spatially closer to the first source (e.g., source S1) than any other detector included in the second plurality of detectors. In this configuration, preventing the light pulses of the second light pulse sequence from temporally overlapping with the light pulses of the first light pulse sequence is configured to cause the first and second detectors to each detect photons from the first light pulse sequence during a first time period that follows each of the light pulses of the first light pulse sequence, and detect photons from the second light pulse sequence during a second time period that follows each of the light pulses of the second light pulse sequence, and that does not temporally overlap with the first time period.

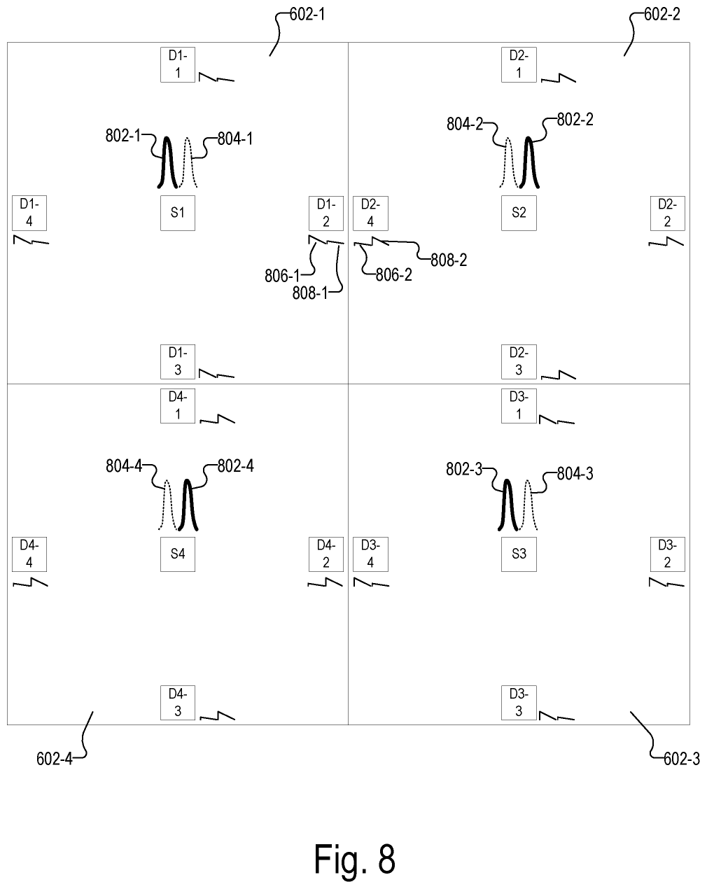

To illustrate the foregoing example, FIG. 8 shows an exemplary time division multiplexing heuristic that may be performed by control circuit 204 with respect to the implementation of assembly 600 shown in FIG. 6. As shown, each source (e.g., S1, S2, S3, and S4) is configured to emit a light pulse 802 during one of two sequential non-overlapping time slots. In particular, source S1 is configured to emit a light pulse 802-1 during the first time slot, but not during the second time slot (as illustrated by dashed lines showing second light pulse 804-1). Source S2 is configured to emit a light pulse 802-2 during the second time slot, but not during the first time slot (as illustrated by dashed lines showing the first light pulse 804-2). Source S3 is configured to emit a light pulse 802-3 during the first time slot, but not during the second time slot (as illustrated by dashed lines showing the second light pulse 804-3). Source S4 is configured to emit a light pulse 802-4 during the second time slot, but not during the first time slot (as illustrated by dashed lines showing the first light pulse 804-4). This pattern of light pulse emission by sources S1-S4 may be repeated over a predetermined amount of time.

In this configuration, the light pulses emitted by sources of neighboring modules (e.g., directly neighboring modules, such as modules 602-1 and 602-2 or modules 602-1 and 602-4) do not overlap and interfere with each other. To illustrate, FIG. 8 shows responses (e.g., responses 806-1 and 806-2 and responses 808-1 and 808-2) detected by the detectors of each of the modules 602 during the time slots associated with the emission of the light pulses 802. These responses may be detected immediately after each of the light pulses 802 are emitted. In some examples, light pulses 802 may be timed so as to not overlap with measurement time windows corresponding to each of the light pulses 802.

For example, detector D1-2 detects response 806-1 in response to the emission of light pulse 802-1 during the first time slot and response 808-1 in response to the emission of light pulse 802-2 during the second time slot. Response 806-1 is shown to be greater in amplitude than response 808-1 because detector D1-2 is spatially closer to source S1 than to source S2. Likewise, detector D2-4 detects response 806-2 in response to the emission of light pulse 802-1 during the first time slot and response 808-2 in response to the emission of light pulse 802-2 during the second time slot. Response 808-2 is shown to be greater in amplitude than response 806-2 because detector D2-4 is spatially closer to source S2 than to source S1.

Because light pulses 802-1 and 802-2 do not temporally overlap (and also because light pulses 802-1 and 802-4 do not temporally overlap), response 806-1 corresponds only to light pulse 802-1 (assuming that detector D1-2 does not detect photons from any other light pulse, such as light pulse 802-3, during the first time slot). Likewise, response 808-2 corresponds only to light pulse 802-2. In this manner, the signal-to-noise ratio of responses 806-1 and 808-2 may be optimized.

Moreover, because responses 806-1 and 808-1 are isolated, detector D1-2 may be configured to provide more useful information (e.g., information represented by both of these responses) than configurations where responses 806-1 and 808-1 are not isolated.

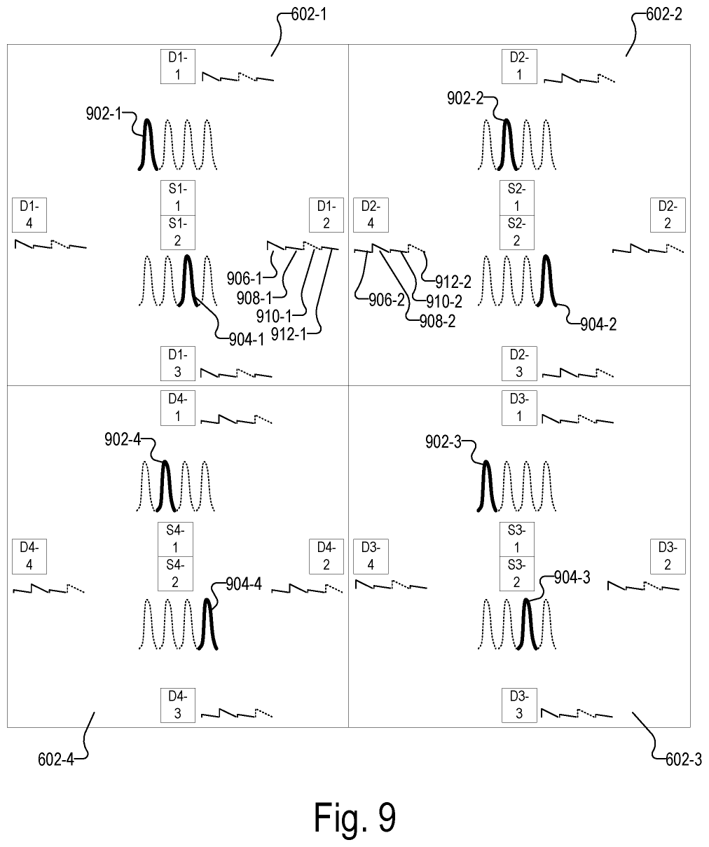

Continuing with the example above with respect to the optical measurement system that includes the first and second wearable modules, the first wearable module may further include a third source (e.g., source S1-2 as shown in FIG. 9) configured to emit a third light pulse sequence comprising a plurality of light pulses each having a second wavelength different than the first wavelength. The third light pulse sequence is time interleaved with the first and second light pulse sequences. The first plurality of detectors is configured to detect photons from the third light pulse sequence.

The second wearable module may further include a fourth source (e.g., source S2-2, shown in FIG. 9) configured to emit a fourth light pulse sequence comprising a plurality of light pulses each having the second wavelength. The fourth light pulse sequence is time interleaved with the first, second, and third light pulse sequences. The second plurality of detectors is configured to detect photons from the fourth light pulse sequence.

In this configuration, the control circuit is configured to prevent the light pulses of the fourth light pulse sequence from temporally overlapping with the light pulses of the first, second, and third light pulse sequences and the light pulses of the third light pulse sequence from temporally overlapping with the light pulses of the first and second light pulse sequences by directing the third source to begin emitting the third light pulse sequence at a third time that is temporally offset from the first and second times and directing the fourth source to begin emitting the fourth light pulse sequence at a fourth time that is temporally offset from the first, second, and third times. Again, for purposes of this example, it is assumed that the first through fourth light pulse sequences are all emitted at the same pulse frequency.

To illustrate the foregoing example, FIG. 9 shows an exemplary time division multiplexing heuristic that may be performed by control circuit 204 with respect to the implementation 700 of assembly 600 shown in FIG. 7. As shown, each source is configured to emit a light pulse 902 or 904 during one of four sequential non-overlapping time slots. In particular, the sources configured to emit light pulses having the first wavelength (i.e., sources S1-1, S2-1, S3-1, and S4-1) are configured to emit a light pulse 902 during one of the first two time slots of the four sequential non-overlapping time slots and the sources configured to emit light pulses having the second wavelength (i.e., sources S1-2, S2-2, S3-2, and S4-2) are configured to emit a light pulse 904 during one of the last two time slots of the four sequential non-overlapping time slots.

To illustrate, source S1-1 is configured to emit a light pulse 902-1 during the first time slot, but not during the second through fourth time slots (as illustrated by the dashed lines in pulse shape by light pulse 902-1). Source S2-1 is configured to emit a light pulse 902-2 during the second time slot, but not during the first, third, and fourth time slots (as illustrated by the dashed lines in pulse shape by light pulse 902-2). Source S3-1 is configured to emit a light pulse 902-3 during the first time slot, but not during the second through fourth time slots (as illustrated by the dashed lines in pulse shape by light pulse 902-3). Source S4-1 is configured to emit a light pulse 902-4 during the second time slot, but not during the first, third, and fourth time slots (as illustrated by the dashed lines in pulse shape by light pulse 902-3). This pattern of light pulse emission by sources S1-1, S2-1, S3-1, and S4-1 may be repeated over a predetermined amount of time.

Likewise, source S1-2 is configured to emit a light pulse 904-1 during the third time slot, but not during the first, second, and fourth time slots (as illustrated by the dashed lines in pulse shape by light pulse 904-1). Source S2-2 is configured to emit a light pulse 904-2 during the fourth time slot, but not during the first through third time slots (as illustrated by the dashed lines in pulse shape by light pulse 904-2). Source S3-2 is configured to emit a light pulse 904-3 during the third time slot, but not during the first, second, and fourth time slots (as illustrated by the dashed lines in pulse shape by light pulse 904-3). Source S4-2 is configured to emit a light pulse 904-4 during the fourth time slot, but not during the first through third time slots (as illustrated by the dashed lines in pulse shape by light pulse 904-4). This pattern of light pulse emission by sources S1-2, S2-2, S3-2, and S4-2 may be repeated over a predetermined amount of time.

In this configuration, the light pulses emitted by sources of neighboring modules (e.g., directly neighboring modules, such as modules 602-1 and 602-2 or modules 602-1 and 602-4) do not overlap and interfere with each other. Moreover, the light pulses emitted by sources on the same module (e.g., sources S1-1 and S1-2) do not overlap and interfere with each other.

To illustrate, FIG. 9 shows responses (e.g., responses 906-1 and 906-2, responses 908-1 and 908-2, responses 910-1 and 910-2, and responses 912-1 and 912-2) detected by detectors of each of the modules 602 during the time slots associated with the emission of the light pulses 902 and 904. These responses may be detected immediately after each of the light pulses 902 and 904 are emitted.

For example, detector D1-2 detects response 906-1 in response to the emission of light pulse 902-1 during the first time slot, response 908-1 in response to the emission of light pulse 902-2 during the second time slot, response 910-1 in response to the emission of light pulse 904-1 during the third time slot, and response 912-1 in response to the emission of light pulse 904-2 during the fourth time slot.

Because light pulses 902-1 and 902-2 do not temporally overlap (and because light pulses 902-1 and 902-4 do not temporally overlap), response 906-1 corresponds only to light pulse 902-1 (assuming that detector D1-2 does not detect photons from any other light pulse, such as light pulse 902-3, during the first time slot). Likewise, response 908-2 corresponds only to light pulse 902-2. In this manner, the signal-to-noise ratio of responses 906-1 and 908-2 may be optimized.

Because light pulses 904-1 and 904-2 do not temporally overlap (and because light pulses 904-1 and 904-4 do not temporally overlap), response 910-1 corresponds only to light pulse 904-1 (assuming that detector D1-2 does not detect photons from any other light pulse, such as light pulse 904-3, during the third time slot). Likewise, response 912-2 corresponds only to light pulse 904-2. In this manner, the signal-to-noise ratio of responses 910-1 and 912-2 may be optimized.

Additionally or alternatively, a control circuit (e.g., control circuit 204 or any other suitable controller) may be configured to implement frequency division multiplexing by controlling a timing of the light pulse sequences emitted by each of the sources within assembly 600.

To illustrate, an optical measurement system may include first and second wearable modules (e.g., modules 602-1 and 602-2). The first wearable module may include a first source (e.g., source S1) configured to emit a first light pulse sequence comprising a plurality of light pulses and a first plurality of detectors (e.g., detectors D1-1, D1-2, D1-3, and D1-4) configured to detect photons from the first light pulse sequence. The second wearable module may likewise include a second source (e.g., source S2) configured to emit a second light pulse sequence comprising a plurality of light pulses and that is time interleaved with the first light pulse sequence and a second plurality of detectors (e.g., detectors D2-1, D2-2, D2-3, and D2-4) configured to detect photons from the second light pulse sequence. A control circuit is configured to implement frequency division multiplexing by directing the first source to emit the first light pulse sequence at a first pulse frequency and directing the second source to emit the second light pulse sequence at a second pulse frequency that is offset with respect to the first pulse frequency.

In frequency division multiplexing, the pulse frequencies from direct neighboring modules (e.g., modules 602-1 and 602-2 or modules 602-1 and 602-4) are slightly offset by a very small amount. Since the detector response is averaged over many cycles, the probability of directly overlapping light pulse repetition is relatively small and becomes essential part of the background noise. Compared to time division multiplexing, the data collection can be increased (e.g., doubled) since one source in each module can fire in every cycle.

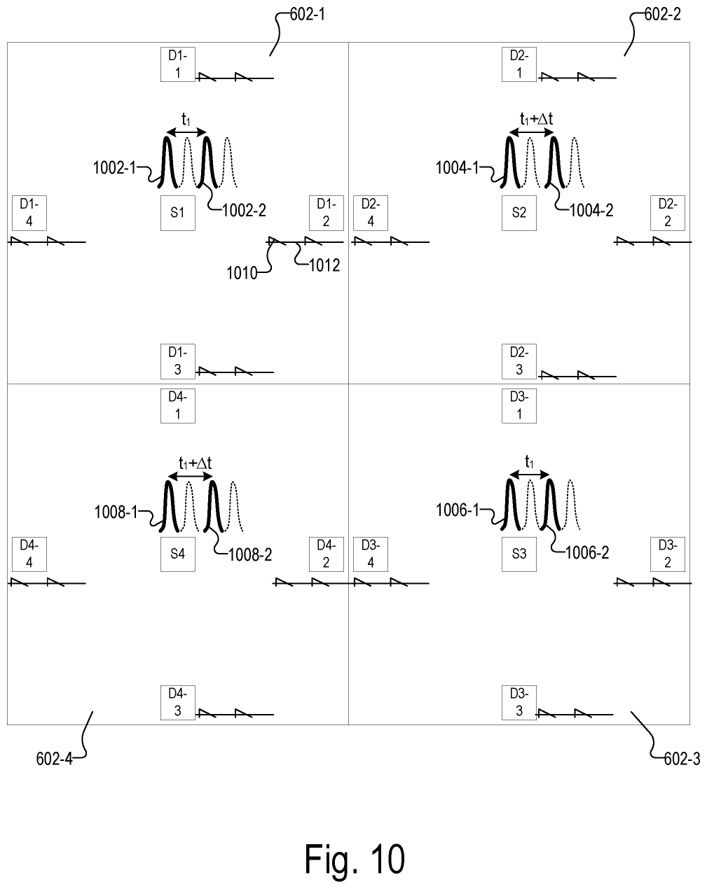

To illustrate the foregoing example, FIG. 10 shows an exemplary frequency division multiplexing heuristic that may be performed by control circuit 204 with respect to the implementation of assembly 600 shown in FIG. 6. As shown, each source is configured to emit a sequence of light pulses including first and second light pulses. For example, source S1 is configured to emit first and second light pulses 1002-1 and 1002-2, source S2 is configured to emit first and second light pulses 1004-1 and 1004-2, source S3 is configured to emit first and second light pulses 1006-1 and 1006-2, and source S4 is configured to emit first and second light pulses 1008-1 and 1008-2. A control circuit is configured to direct sources of neighboring modules 602 to emit the light pulses at slightly different pulse frequencies. For example, the time between light pulses 1002-1 and 1002-2 and between light pulses 1006-1 and 1006-2 is t₁, while the time between light pulses 1004-1 and 1004-2 and between light pulses 1008-1 and 1008-2 is t₁+Δt. This offset (i.e., Δt) may be any suitable amount of time, such as a value less than a time period of one of the light pulses in any of the light pulse sequences.

FIG. 10 shows responses (e.g., response 1010) detected by detectors of each of the modules 602 during time slots associated with the emission of the light pulses 1002-1008. These responses may be detected immediately after each of the light pulses are emitted. As shown by a horizontal line (e.g., horizontal line 1012), a noise floor associated with the responses may exist for each of the responses due to nearest neighbor interference, but signal processing circuitry may be configured to filter such noise out as may serve a particular implementation.

Continuing with the example above with respect to the optical measurement system that includes the first and second wearable modules, the first wearable module may further include a third source (e.g., source S1-2) configured to emit a third light pulse sequence comprising a plurality of light pulses each having a second wavelength different than the first wavelength. The third light pulse sequence is time interleaved with the first and second light pulse sequences. The first plurality of detectors is configured to detect photons from the third light pulse sequence.

The second wearable module may further include a fourth source (e.g., source S2-2) configured to emit a fourth light pulse sequence comprising a plurality of light pulses each having the second wavelength. The fourth light pulse sequence is time interleaved with the first, second, and third light pulse sequences. The second plurality of detectors is configured to detect photons from the fourth light pulse sequence.

In this configuration, the control circuit is further configured to perform frequency division multiplexing by directing the third source to emit the third light pulse sequence at a first pulse frequency and directing the fourth source to emit the fourth light pulse sequence at the second pulse frequency.

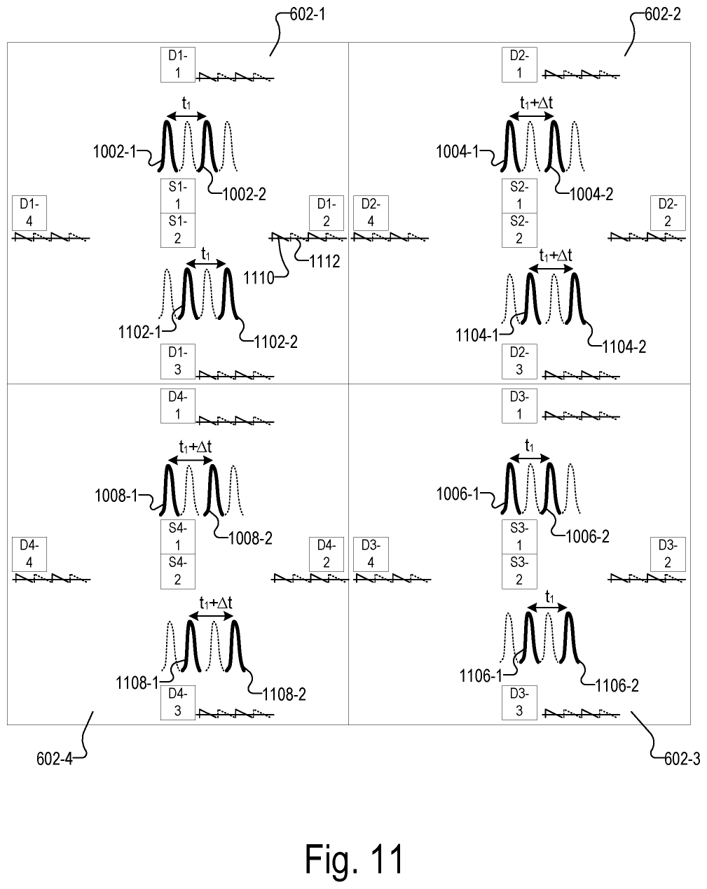

To illustrate the foregoing example, FIG. 11 shows an exemplary frequency division multiplexing heuristic that may be performed by control circuit 204 with respect to the implementation 700 of assembly 600 shown in FIG. 7. As shown, FIG. 11 is similar to FIG. 10 in that sources S1-1, S2-1, S3-1, and S4-1 emit light pulses 1002, 1004, 1006, and 1008, respectively. However, in FIG. 11, source S1-2 is configured to emit first and second light pulses 1102-1 and 1102-2, source S2-2 is configured to emit first and second light pulses 1104-1 and 1104-2, source S3-2 is configured to emit first and second light pulses 1106-1 and 1106-2, and source S4-2 is configured to emit first and second light pulses 1108-1 and 1108-2. A control circuit is configured to direct sources of neighboring modules 602 to emit these light pulses at slightly different pulse frequencies. For example, the time between light pulses 1102-1 and 1102-2 and between light pulses 1106-1 and 1106-2 is t₁, while the time between light pulses 1104-1 and 1104-2 and between light pulses 1108-1 and 1108-2 is t₁+Δt.

FIG. 11 shows responses (e.g., response 1110) detected by detectors of each of the modules 602 during time slots associated with the emission of the light pulses 1002-1008 and 1102-1108. These responses may be detected immediately after each of the light pulses are emitted. As shown by a horizontal line (e.g., horizontal line 1112), a noise floor associated with the responses may exist for each of the responses due to nearest neighbor interference, but signal processing circuitry may be configured to filter such noise out as may serve a particular implementation.

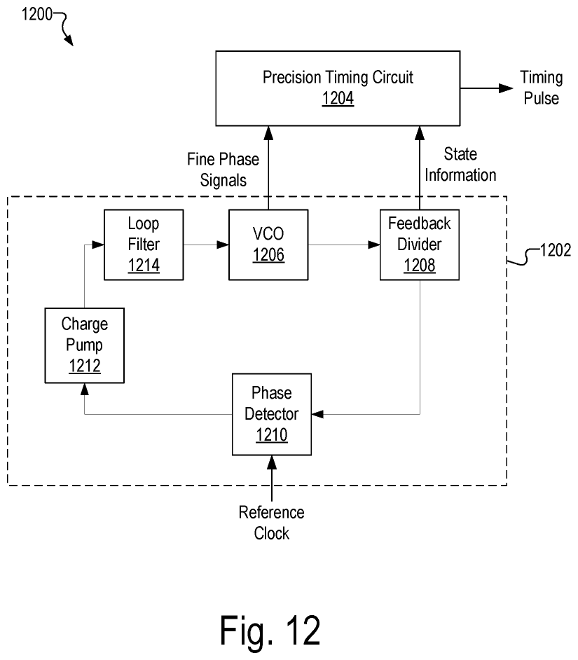

A timing and/or temporal position of the light pulses may be precisely controlled (e.g., set) in any suitable manner. For example, FIG. 12 illustrates an exemplary PLL circuit based architecture 1200 that may be included within optical measurement system 100 to generate and set a temporal position (e.g., of a rising edge and/or of a falling edge) of a timing pulse that may be used to set a temporal position of any of the light pulses described herein. As shown, architecture 1200 includes a PLL circuit 1202 communicatively coupled to a precision timing circuit 1204. PLL circuit 1202 includes a VCO 1206, a feedback divider 1208, a phase detector 1210, a charge pump 1212, and a loop filter 1214 connected in a feedback loop configuration. Phase detector 1210 may receive a reference clock as an input such that PLL circuit 1202 has a PLL feedback period defined by the reference clock. The reference clock may have any suitable frequency, such as any frequency between 1 MHz and 200 MHz.

VCO 1206 may be implemented by any suitable combination of circuitry (e.g., a differential multi-stage gated ring oscillator (GRO) circuit) and is configured to lock to the reference clock (i.e., to a multiple of a frequency of the reference clock). To that end, VCO 1206 may include a plurality of stages configured to output a plurality of fine phase signals each having a different phase and uniformly distributed in time. In some examples, each stage may output two fine phase signals that have complimentary phases. VCO 1206 may include any suitable number of stages configured to output any suitable number of fine phase signals (e.g., eight stages that output sixteen fine phase signals). The duration of a fine phase signal pulse depends on the oscillator frequency of VCO 1206 and the total number of fine phase signals. For example, if the oscillator frequency is 1 gigahertz (GHz) and the total number of fine phase signals is sixteen, the duration of a pulse included in a fine phase signal is 1 GHz/16, which is 62.5 picoseconds (ps). As described herein, these fine phase signals may provide precision timing circuit 1204 with the ability to adjust a phase (i.e., temporal position) of a timing pulse with relatively fine resolution.

Feedback divider 1208 is configured to be clocked by a single fine phase signal included in the plurality of fine phase signals output by VCO 1206 and have a plurality of feedback divider states during the PLL feedback period. The number of feedback divider states depends on the oscillator frequency of VCO 1206 and the frequency of the reference clock. For example, if the oscillator frequency is 1 gigahertz (GHz) and the reference clock has a frequency of 50 MHz, the number of feedback divider states is equal to 1 GHz/50 MHz, which is equal to 20 feedback divider states. As described herein, these feedback divider states may provide precision timing circuit 1204 with the ability to adjust a phase (i.e., temporal position) of a timing pulse with relatively course resolution.

Feedback divider 1208 may be implemented by any suitable circuitry. In some alternative examples, feedback divider 1208 is at least partially integrated into precision timing circuit 1204.

As shown, the fine phase signals output by VCO 1206 and state information (e.g., signals and/or data) representative of the feedback divider states within feedback divider 1208 are input into precision timing circuit 1204. Precision timing circuit 1204 may be configured to generate a timing pulse and set, based on a combination of one of the fine phase signals and one of the feedback dividers states, a temporal position of the timing pulse within the PLL feedback period. For example, if there are N total fine phase signals and M total feedback divider states, precision timing circuit 1204 may set the temporal position of the timing pulse to be one of N times M possible temporal positions within the PLL feedback period. To illustrate, if N is 16 and M is 20, and if the duration of a pulse included in a fine phase signal is 62.5 ps, the temporal position of the timing pulse may be set to be one of 320 possible positions in 62.5 ps steps.

The timing pulse generated by precision timing circuit 1204 may be used within optical measurement system 100 in any suitable manner. For example, the timing pulse may be configured to trigger a start (e.g., a rising edge) of an output pulse used by a component within optical measurement system 100. Alternatively, the timing pulse may be configured to trigger an end (e.g., a falling edge) of an output pulse used by a component within optical measurement system 100. Alternatively, the timing pulse itself may be provided for use as an output pulse used by a component within optical measurement system 100. In some examples, precision timing circuit 1204 may generate multiple timing pulses each used for a different purpose within optical measurement system 100. PLL circuit based architecture 1200 is described in more detail in U.S. Provisional Patent Application No. 63/027,011, filed May 19, 2020, and incorporated herein by reference in its entirety.

In some examples, the control circuit is configured to set one or more parameters associated with the time and/or frequency division multiplexing heuristics described herein in accordance with a composition configuration (e.g., a composition vector) that controls an operation of the various sources that may be included in an optical measurement system.

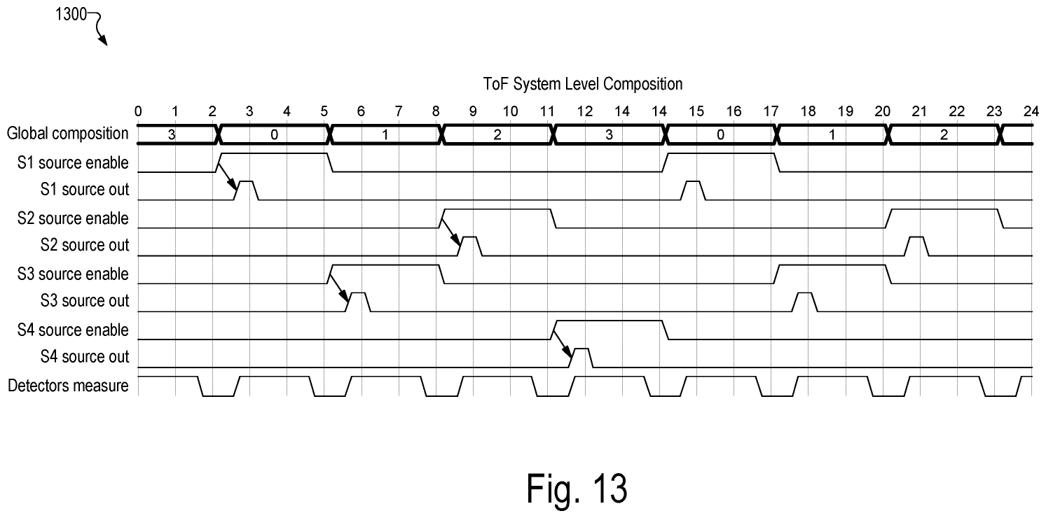

To illustrate, FIG. 13 shows an exemplary composition configuration 1300 that may be used in accordance with the systems, circuits, and methods described herein. Composition configuration 1300 may be used to indicate in which state each of the sources and detectors should be enabled. In this example, source S1 is enabled in composition state 0 while source S2 is enabled in composition state 2, etc. In this example, the composition states are periodic and repeating every 4 states. In principle, this can be programmed so that arbitrary composition state patterns can achieved in order to optimize data collection. When recording the data, the detector then stores these composition values so that each collection has a record of what the configuration for that signal was. The composition states can be programmed globally with each source/detector having a unique enable state (or potentially multiple states).

In some examples, a processor (e.g., processor 108, signal processing circuit 208, and/or any other suitable processor) may receive outputs from the detectors described herein and determine, based on the outputs, a property of a target. For example, the processor may use the outputs to detect a concentration of oxygenated hemoglobin in brain tissue, a concentration of deoxygenated hemoglobin in the brain tissue, and/or any other suitable property of a target that scatters light before photons of the light are detected by the detectors.

As mentioned, optical measurement system 100 may be at least partially wearable by a user. For example, optical measurement system 100 may be implemented by a wearable device configured to be worn by a user (e.g., a head-mountable component configured to be worn on a head of the user). The wearable device may include one or more photodetectors, modules, and/or any of the other components described herein. In some examples, one or more components (e.g., processor 108, controller 112, etc.) may not be included in the wearable device and/or included in a separate wearable device than the wearable device in which the one or more photodetectors are included. In these examples, one or more communication interfaces (e.g., cables, wireless interfaces, etc.) may be used to facilitate communication between the various components.

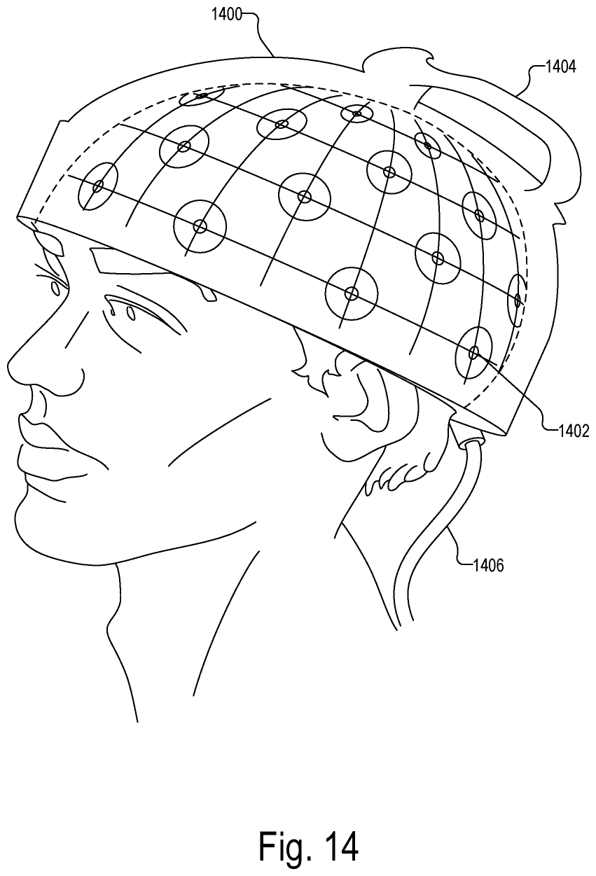

FIGS. 14-19 illustrate embodiments of a wearable device 1400 that includes elements of the optical detection systems described herein. In particular, the wearable devices 1400 shown in FIGS. 14-19 include a plurality of modules 1402, similar to the modules described herein. For example, each module 1402 includes a source and a plurality of detectors. The wearable devices 1400 may each also include a controller (e.g., controller 112) and a processor (e.g., processor 108) and/or be communicatively connected to a controller and processor. In general, wearable device 1400 may be implemented by any suitable headgear and/or clothing article configured to be worn by a user. The headgear and/or clothing article may include batteries, cables, and/or other peripherals for the components of the optical measurement systems described herein.

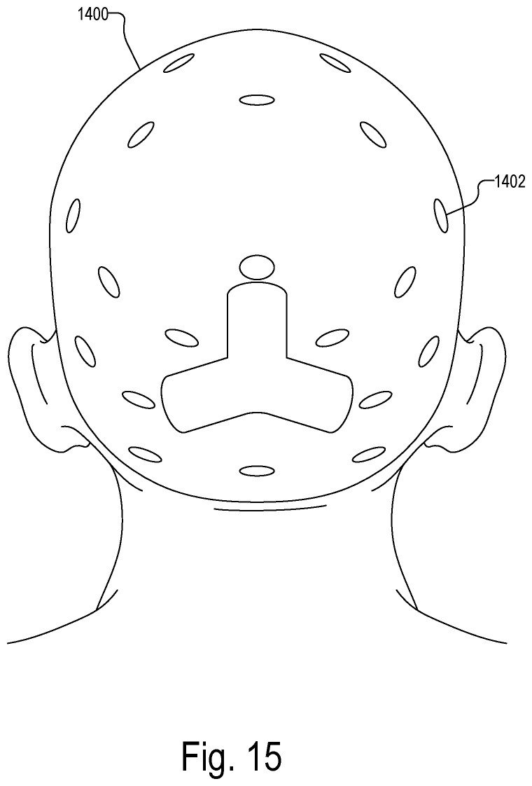

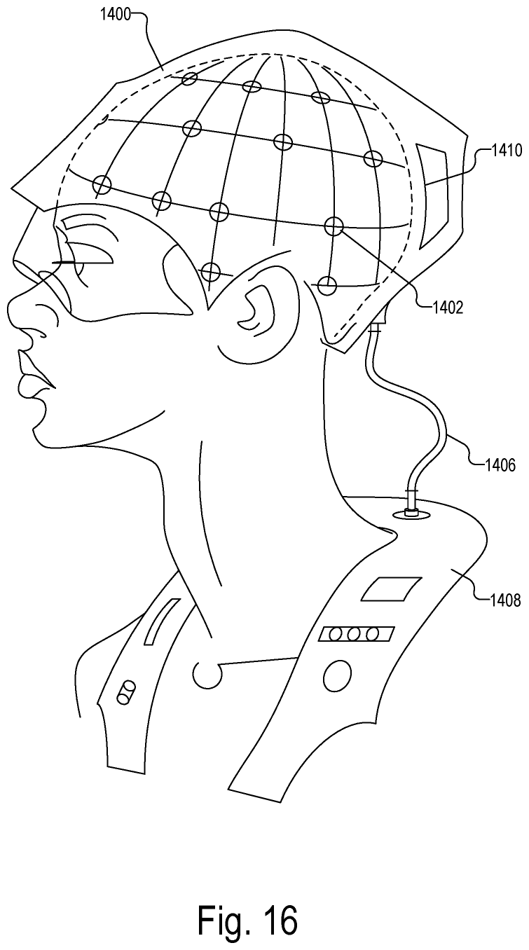

FIG. 14 illustrates an embodiment of a wearable device 1400 in the form of a helmet with a handle 1404. A cable 1406 extends from the wearable device 1400 for attachment to a battery or hub (with components such as a processor or the like). FIG. 15 illustrates another embodiment of a wearable device 1400 in the form of a helmet showing a back view. FIG. 16 illustrates a third embodiment of a wearable device 1400 in the form of a helmet with the cable 1406 leading to a wearable garment 1408 (such as a vest or partial vest) that can include a battery or a hub. Alternatively or additionally, the wearable device 1400 can include a crest 1410 or other protrusion for placement of the hub or battery.

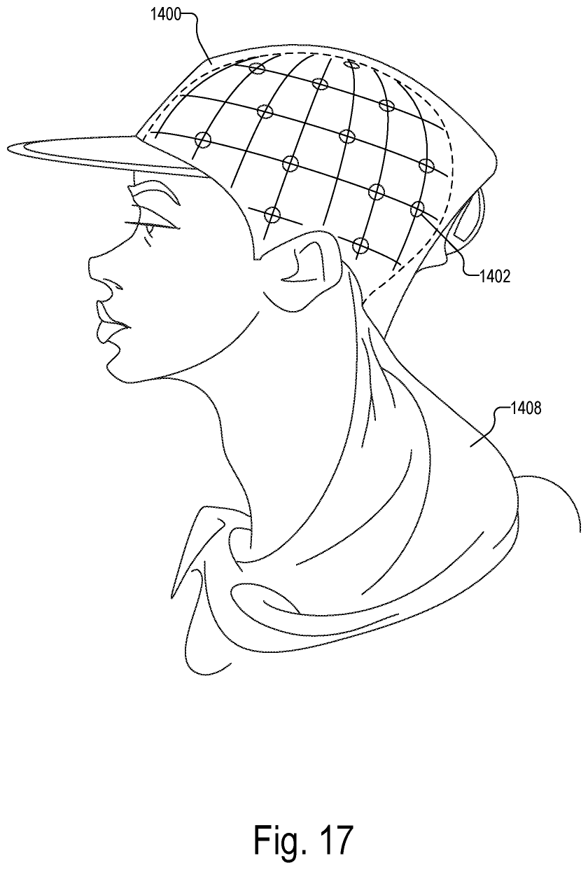

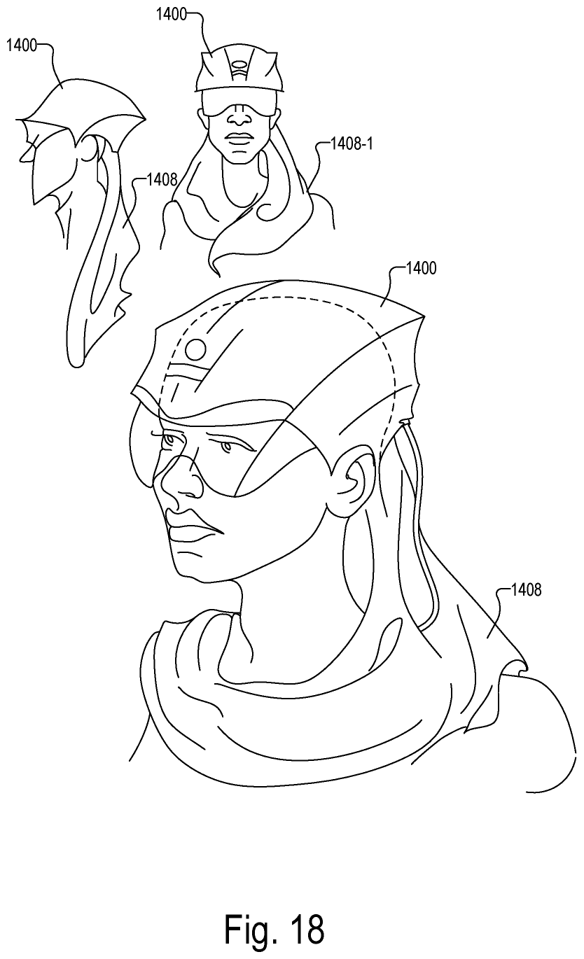

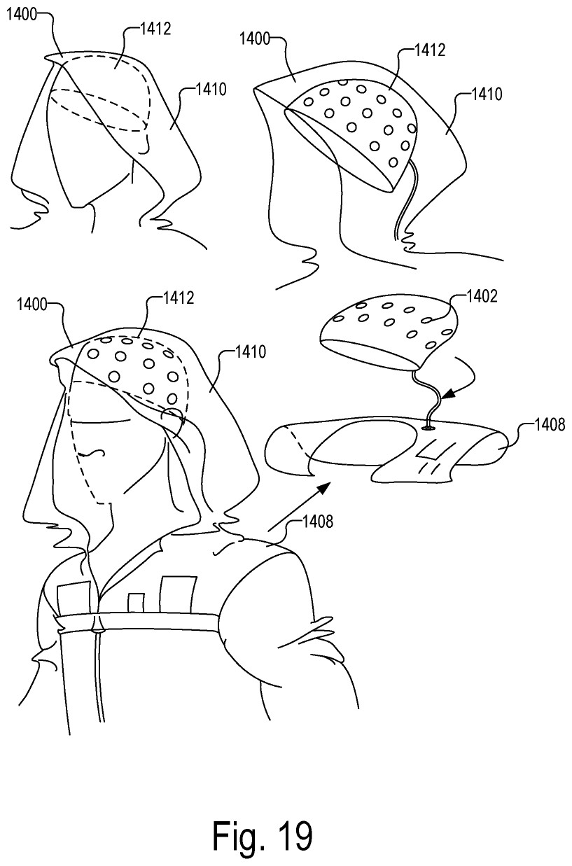

FIG. 17 illustrates another embodiment of a wearable device 1400 in the form of a cap with a wearable garment 1408 in the form of a scarf that may contain or conceal a cable, battery, and/or hub. FIG. 18 illustrates additional embodiments of a wearable device 1400 in the form of a helmet with a one-piece scarf 1408 or two-piece scarf 1408-1. FIG. 19 illustrates an embodiment of a wearable device 1400 that includes a hood 1410 and a beanie 1412 which contains the modules 1402, as well as a wearable garment 1408 that may contain a battery or hub.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

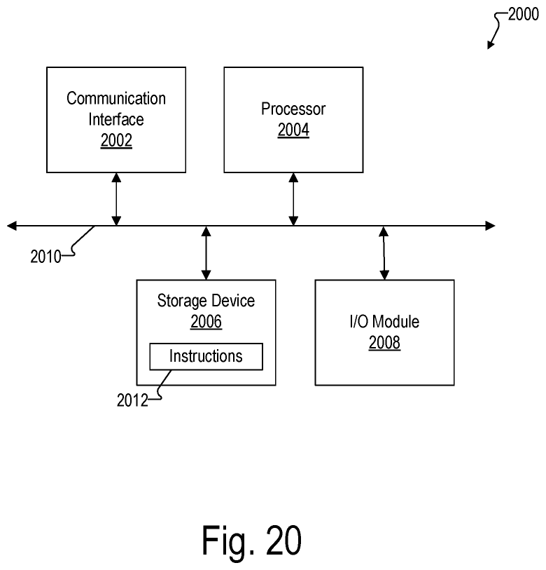

FIG. 20 illustrates an exemplary computing device 2000 that may be specifically configured to perform one or more of the processes described herein. Any of the systems, units, computing devices, and/or other components described herein may be implemented by computing device 2000.

As shown in FIG. 20, computing device 2000 may include a communication interface 2002, a processor 2004, a storage device 2006, and an input/output (“I/O”) module 2008 communicatively connected one to another via a communication infrastructure 2010. While an exemplary computing device 2000 is shown in FIG. 20, the components illustrated in FIG. 20 are not intended to be limiting. Additional or alternative components may be used in other embodiments. Components of computing device 2000 shown in FIG. 20 will now be described in additional detail.

Communication interface 2002 may be configured to communicate with one or more computing devices. Examples of communication interface 2002 include, without limitation, a wired network interface (such as a network interface card), a wireless network interface (such as a wireless network interface card), a modem, an audio/video connection, and any other suitable interface.

Processor 2004 generally represents any type or form of processing unit capable of processing data and/or interpreting, executing, and/or directing execution of one or more of the instructions, processes, and/or operations described herein. Processor 2004 may perform operations by executing computer-executable instructions 2012 (e.g., an application, software, code, and/or other executable data instance) stored in storage device 2006.

Storage device 2006 may include one or more data storage media, devices, or configurations and may employ any type, form, and combination of data storage media and/or device. For example, storage device 2006 may include, but is not limited to, any combination of the non-volatile media and/or volatile media described herein. Electronic data, including data described herein, may be temporarily and/or permanently stored in storage device 2006. For example, data representative of computer-executable instructions 2012 configured to direct processor 2004 to perform any of the operations described herein may be stored within storage device 2006. In some examples, data may be arranged in one or more databases residing within storage device 2006.

I/O module 2008 may include one or more I/O modules configured to receive user input and provide user output. I/O module 2008 may include any hardware, firmware, software, or combination thereof supportive of input and output capabilities. For example, I/O module 2008 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 2008 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 2008 is configured to provide graphical data to a display for presentation to a user. The graphical data may be representative of one or more graphical user interfaces and/or any other graphical content as may serve a particular implementation.

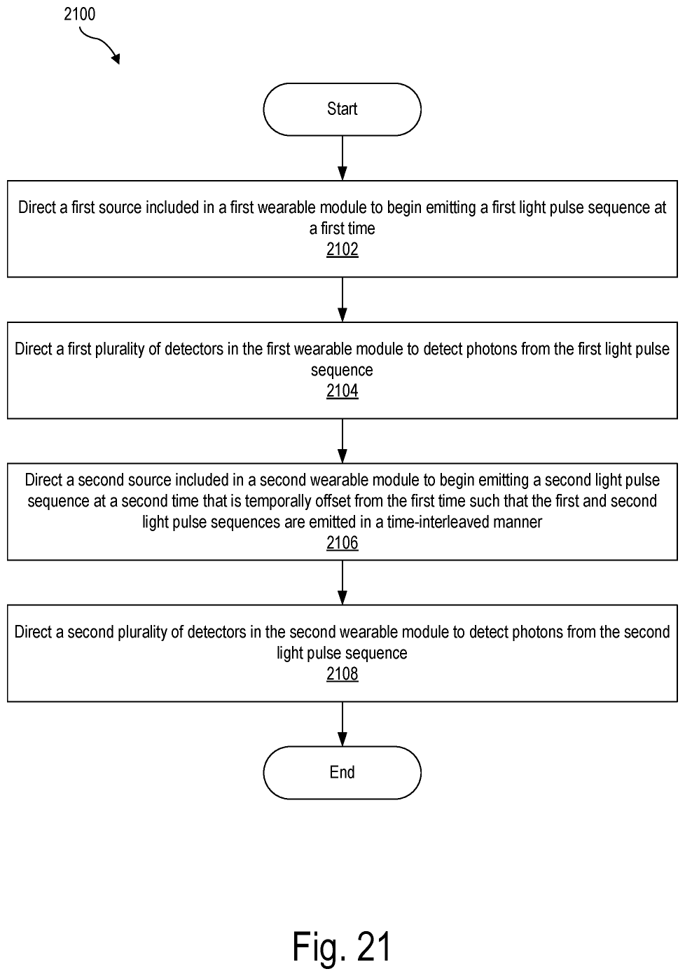

FIG. 21 illustrates an exemplary method 2100 that may be performed by a control circuit. While FIG. 21 illustrates exemplary operations according to one embodiment, other embodiments may omit, add to, reorder, and/or modify any of the operations shown in FIG. 21. Each of the operations shown in FIG. 21 may be performed in any of the ways described herein.

In operation 2102, a control circuit directs a first source included in a first wearable module to begin emitting a first light pulse sequence at a first time.

In operation 2104, the control circuit directs a first plurality of detectors in the first wearable module to detect photons from the first light pulse sequence.

In operation 2106, the control circuit directs a second source included in a second wearable module to begin emitting a second light pulse sequence at a second time that is temporally offset from the first time such that the first and second light pulse sequences are emitted in a time-interleaved manner.

In operation 2108, the control circuit directs a second plurality of detectors in the second wearable module to detect photons from the second light pulse sequence.

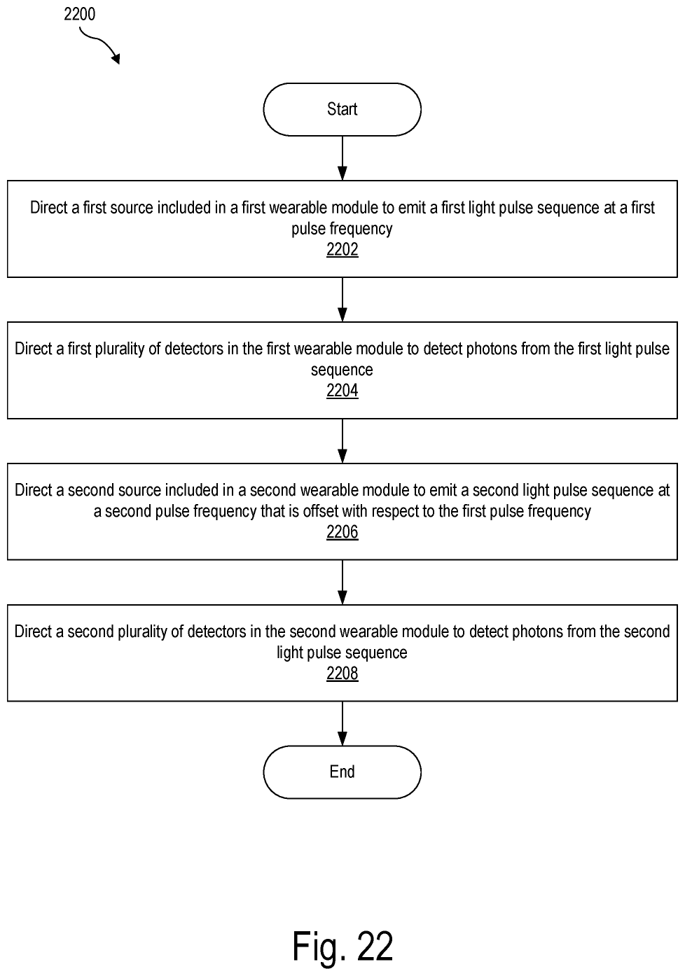

FIG. 22 illustrates an exemplary method 2200 that may be performed by a control circuit. While FIG. 22 illustrates exemplary operations according to one embodiment, other embodiments may omit, add to, reorder, and/or modify any of the operations shown in FIG. 22. Each of the operations shown in FIG. 22 may be performed in any of the ways described herein.

In operation 2202, a control circuit directs a first source included in a first wearable module to emit a first light pulse sequence at a first pulse frequency.

In operation 2204, the control circuit directs a first plurality of detectors in the first wearable module to detect photons from the first light pulse sequence.

In operation 2206, the control circuit directs a second source included in a second wearable module to emit a second light pulse sequence at a second pulse frequency that is offset with respect to the first pulse frequency.

In operation 2208, the control circuit directs a second plurality of detectors in the second wearable module to detect photons from the second light pulse sequence.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. An optical measurement system comprising:
a first wearable module comprising
a first source configured to emit a first light pulse sequence comprising a plurality of light pulses, and
a first plurality of detectors configured to detect photons from the first light pulse sequence;
a second wearable module comprising
a second source configured to emit a second light pulse sequence comprising a plurality of light pulses and that is time interleaved with the first light pulse sequence, and
a second plurality of detectors configured to detect photons from the second light pulse sequence;
a control circuit configured to prevent the light pulses of the second light pulse sequence from temporally overlapping with the light pulses of the first light pulse sequence by
directing the first source to begin emitting the first light pulse sequence at a first time, and
directing the second source to begin emitting the second light pulse sequence at a second time that is temporally offset from the first time.

2. The optical measurement system of claim 1, wherein:
the first plurality of detectors comprises a first detector spatially closer to the second source than any other detector included in the first plurality of detectors;
the second plurality of detectors comprises a second detector spatially closer to the first source than any other detector included in the second plurality of detectors;
the preventing of the light pulses of the second light pulse sequence from temporally overlapping with the light pulses of the first light pulse sequence is configured to cause the first and second detectors to each detect photons from the first light pulse sequence during a first time period that follows each of the light pulses of the first light pulse sequence and detect photons from the second light pulse sequence during a second time period that follows each of the light pulses of the second light pulse sequence and that does not temporally overlap with the first time period.

3. The optical measurement system of claim 1, wherein the first and second light pulse sequences are each emitted at a same pulse frequency.

4. The optical measurement system of claim 1, wherein:
the first source is configured to emit the light pulses of the first light pulse sequence at a first wavelength; and
the second source is configured to emit the light pulses of the second light pulse sequence at the first wavelength.

5. The optical measurement system of claim 4, wherein:
the first wearable module further comprises a third source configured to emit a third light pulse sequence comprising a plurality of light pulses each having a second wavelength, the third light pulse sequence time interleaved with the first and second light pulse sequences;
the first plurality of detectors is configured to detect photons from the third light pulse sequence;
the second wearable module further comprises a fourth source configured to emit a fourth light pulse sequence comprising a plurality of light pulses each having the second wavelength, the fourth light pulse sequence time interleaved with the first, second, and third light pulse sequences;
the second plurality of detectors is configured to detect photons from the fourth light pulse sequence; and
the control circuit is configured to prevent the light pulses of the fourth light pulse sequence from temporally overlapping with the light pulses of the first, second, and third light pulse sequences and the light pulses of the third light pulse sequence from temporally overlapping with the light pulses of the first and second light pulse sequences by
directing the third source to begin emitting the third light pulse sequence at a third time that is temporally offset from the first and second times, and
directing the fourth source to begin emitting the fourth light pulse sequence at a fourth time that is temporally offset from the first, second, and third times.

6. The optical measurement system of claim 1, wherein:
the first and second plurality of detectors are configured to detect photons from one or more of the first or second light pulse sequences after the first and second light pulse sequences are scattered by a target within a body; and
the optical measurement system further comprises a processor configured to:
receive outputs from the first and second plurality of detectors, and
determine, based on the outputs, a property of the target.

7. The optical measurement system of claim 6, wherein the property comprises one or more of a concentration of oxygenated hemoglobin in brain tissue or a concentration of deoxygenated hemoglobin in the brain tissue.

8. The optical measurement system of claim 1, further comprising a head-mountable component configured to be worn on a head of a user, wherein the first and second wearable modules are included in the head-mountable component.

9. The optical measurement system of claim 8, wherein the control circuit is included in the head-mountable component.

10. The optical measurement system of claim 1, wherein each detector included in the first and second plurality of detectors comprises a plurality of time-resolved single photon photodetectors.

11. The optical measurement system of claim 10, wherein the plurality of time-resolved single photon photodetectors comprise a plurality of single-photon avalanche diode (SPAD) circuits.

12. The optical measurement system of claim 1, wherein the control circuit is configured to set the first and second times in accordance with a composition configuration that controls an operation of the first and second sources.

13. An optical measurement system comprising:
a first wearable module comprising
a first source configured to emit a first light pulse sequence comprising a plurality of light pulses, and
a first plurality of detectors configured to detect photons from the first light pulse sequence;
a second wearable module comprising
a second source configured to emit a second light pulse sequence comprising a plurality of light pulses and that is time interleaved with the first light pulse sequence, and
a second plurality of detectors configured to detect photons from the second light pulse sequence; and
a control circuit configured to
direct the first source to emit the first light pulse sequence at a first pulse frequency; and
direct the second source to emit the second light pulse sequence at a second pulse frequency that is offset with respect to the first pulse frequency.

14. The optical measurement system of claim 13, wherein the second pulse frequency is offset with respect to the first pulse frequency by less than a time period of one of the light pulses in the first light pulse sequence.

15. The optical measurement system of claim 13, wherein:
the first source is configured to emit the light pulses of the first light pulse sequence at a first wavelength; and
the second source is configured to emit the light pulses of the second light pulse sequence at the first wavelength.

16. The optical measurement system of claim 15, wherein:
the first wearable module further comprises a third source configured to emit a third light pulse sequence comprising a plurality of light pulses each having a second wavelength, the third light pulse sequence time interleaved with the first and second light pulse sequences;
the first plurality of detectors is configured to detect photons from the third light pulse sequence;
the second wearable module further comprises a fourth source configured to emit a fourth light pulse sequence comprising a plurality of light pulses each having the second wavelength, the fourth light pulse sequence time interleaved with the first, second, and third light pulse sequences;
the second plurality of detectors is configured to detect photons from the fourth light pulse sequence; and
the control circuit is further configured to
direct the third source to emit the third light pulse sequence at a first pulse frequency; and
direct the fourth source to emit the fourth light pulse sequence at the second pulse frequency.

17. The optical measurement system of claim 13, wherein:
the first and second plurality of detectors are configured to detect photons from one or more of the first or second light pulse sequences after the first and second light pulse sequences are scattered by a target within a body; and
the optical measurement system further comprises a processor configured to:
receive outputs from the first and second plurality of detectors, and
determine, based on the outputs, a property of the target.

18. The optical measurement system of claim 17, wherein the property comprises one or more of a concentration of oxygenated hemoglobin in brain tissue or a concentration of deoxygenated hemoglobin in the brain tissue.

19. The optical measurement system of claim 13, further comprising a head-mountable component configured to be worn on a head of a user, wherein the first and second wearable modules are included in the head-mountable component.

20. The optical measurement system of claim 19, wherein the control circuit is included in the head-mountable component.

21. The optical measurement system of claim 13, wherein each detector included in the first and second plurality of detectors comprises a plurality of time-resolved single photon photodetectors.

22. The optical measurement system of claim 21, wherein the plurality of time-resolved single photon photodetectors comprise a plurality of single-photon avalanche diode (SPAD) circuits.

23. The optical measurement system of claim 13, wherein the control circuit is configured to set the first and second times in accordance with a composition configuration that controls an operation of the first and second sources.

24-30. (canceled)
